(19) United States
(12) Patent Application Publication
(10) Pub. No.: US 2009/0287048 A1
(43) Pub. Date: Nov. 19, 2009

(54) Method and apparatus for imaging within a living body

(75) Inventors: Stephen C. Jacobson, Salt Lake City, UT (US); David Wells, Toronto (CA)

Correspondence Address: THORPE NORTH & WESTERN, LLP., P.O. Box 1219, SANDY, UT, 84091-1219, US

(73) Assignee: Sterling LC

(21) Appl. No.: 12/152,730

(22) Filed: May 16, 2008

(51) Int. Cl. A61B 1/04

(52) U.S. Cl. 600/109

(57) ABSTRACT

A method and apparatus for imaging within a living body is described. The method includes directing a micro-guidewire along a primary path of the living body, the micro-guidewire having an imaging device including a SSID with an imaging array and a GRIN lens optically coupled to the imaging array. A secondary path can be identified, laterally branching from the primary path, the secondary path being of much smaller dimensions than the primary path. The distal end of the micro-guidewire can be turned and advanced into the secondary path by applied pressure at a proximal end of the micro-guidewire.

### BACKGROUND

Minimally invasive diagnostic medical procedures are used to assess the interior surfaces of an organ by inserting a tube into the body. Instruments used for such procedures may have a rigid or flexible tube and not only provide an image for visual inspection and photography, but also enable taking biopsies and retrieval of foreign objects. The size of instruments utilized for such procedures has limited the extent that instruments may travel within the body.

### BRIEF DESCRIPTION OF THE DRAWINGS

In the accompanying drawings:

FIG. 1 is a schematic illustration of an exemplary medical imaging system in accordance with principles of one embodiment of the invention;

FIG. 2 is a side view of an exemplary embodiment of the present invention, which is an enlarged view of device 14 of FIG. 1;

FIG. 3 is a perspective view of another exemplary embodiment of the invention;

FIG. 4 is a top view of the device of FIG. 3;

FIG. 5 is a side view of the device of FIG. 3, rotated 90 degrees with respect to FIG. 4;

FIG. 6 is a cross sectional view of another exemplary embodiment of the invention;

FIG. 7 is a cross sectional view of another exemplary embodiment of the invention;

FIG. 8 is a cross sectional view of another exemplary embodiment of the invention in a first configuration;

FIG. 9 is a cross sectional view of the device of FIG. 8 in a second position view;

FIG. 10 is a perspective view of an SSID optically coupled to a GRIN lens;

FIG. 11 is a perspective view of an exemplary embodiment of an SSID and multiple GRIN lens positioned in an array;

FIG. 12 is a perspective view of another exemplary embodiment of an SSID and multiple GRIN lens positioned in an array;

FIG. 13 is a side view of multiple microcameras positioned along an umbilical as an array;

FIG. 14 is plan view along the optical axis of an exemplary color filter insert that can be used with imagine devices in accordance with principles of the invention;

FIG. 15 is a first side view of the color filter insert of FIG. 14;

FIG. 16 is a second side view of the color filter insert of FIG. 14, taken at 90 degrees with respect to FIG. 15;

FIG. 17 is a schematic side view representation of another exemplary embodiment having a color filter insert of FIG. 14 inserted therein;

FIG. 18 is a schematic side view representation of another exemplary embodiment having a fiber optic inserted therein;

FIG. 19 is a schematic illustration of an exemplary miniature imaging system for imaging the gastrointestinal tract in accordance with one embodiment of the invention;

FIG. 20 is a schematic illustration of an exemplary miniature imaging system for imaging the pancreas and gallbladder in accordance with one embodiment of the invention;

FIG. 21 is a schematic illustration of an exemplary miniature imaging system for imaging the colon in accordance with one embodiment of the invention;

FIG. 22 is a schematic illustration of an exemplary miniature imaging system for imaging the lungs in accordance with one embodiment of the invention;

FIG. 23 is a schematic illustration of an exemplary miniature imaging system for imaging the sinuses in accordance with one embodiment of the invention;

FIG. 24 is a schematic illustration of an exemplary miniature imaging system for imaging the kidneys in accordance with one embodiment of the invention;

FIG. 25 is a schematic illustration of an exemplary miniature imaging system for imaging the fallopian tubes and ovaries in accordance with one embodiment of the invention;

FIG. 26 is a schematic illustration of an exemplary miniature imaging system for imaging the appendix in accordance with one embodiment of the invention;

FIG. 27 is a flowchart of a method for a method for imaging within a living body in accordance with one embodiment of the invention; and

FIG. 28 is a schematic illustration of an exemplary miniature imaging system in accordance with one embodiment of the invention;

### DETAILED DESCRIPTION OF THE PREFERRED EMBODIMENT(S)

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

It must be noted that, as used in this specification and the appended claims, singular forms of “a,” “an,” and “the” include plural referents unless the context clearly dictates otherwise.

An “SSID,” “solid state imaging device,” or “SSID chip” in the exemplary embodiments generally comprises an imaging array or pixel array for gathering image data, and can further comprise conductive pads electrically coupled to the imaging array, which facilitates electrical communication therebetween. In one embodiment, the SSID can comprise a silicon or silicon-like substrate or amorphous silicon thin film transistors (TFT) having features typically manufactured therein. Features can include the imaging array, the conductive pads, metal traces, circuitry, etc. Other integrated circuit components can also be present for desired applications. However, it is not required that all of these components be present, as long as there is a means of gathering visual or photon data, and a means of sending that data to provide a visual image or image reconstruction.

The term “umbilical” can include the collection of utilities that operate the SSID or the micro-camera as a whole. Typically, an umbilical includes a conductive line, such as electrical wire(s) or other conductors, for providing power, ground, clock signal, and output signal with respect to the SSID, though not all of these are strictly required. For example, ground can be provided by another means other than through an electrical wire, e.g., to a camera housing such as micromachined tubing, etc. The umbilical can also include other utilities such as a light source, temperature sensors, force sensors, fluid irrigation or aspiration members, pressure sensors, fiber optics, microforceps, material retrieval tools, drug delivery devices, and radiation emitting devices, laser diodes, electric cauterizers, and electric stimulators, for example. Other utilities will also be apparent to those skilled in the art and are thus comprehended by this disclosure.

“GRIN lens” or “graduated refractive index lens” refers to a specialized lens that has a refractive index that is varied radially from a center optical axis to the outer diameter of the lens. In one embodiment, such a lens can be configured in a cylindrical shape, with the optical axis extending from a first flat end to a second flat end. Thus, because of the differing refractive index in a radial direction from the optical axis, a lens of this shape can simulate the affects of a more traditionally shaped lens.

With these definitions in mind, reference will now be made to the accompanying drawings, which illustrate, by way of example, embodiments of the invention.

Small imaging devices can be particularly useful in medical diagnostic and treatment applications. Portions of human anatomy previously viewable only by a surgical procedure can be viewed now by a minimally invasive procedures, provided an imaging device can be made that is small enough to view the target anatomy. Other uses for very small imaging devices are recognized. For example, such devices can be used and are desirable for surveillance applications, for monitoring of conditions and functions within devices, and for size- and weight-critical imaging needs as are present in aerospace applications, to name a few.

While the present invention has applications in these aforementioned fields and others, the medical imaging application can be used to favorably illustrate unique advantages of the invention.

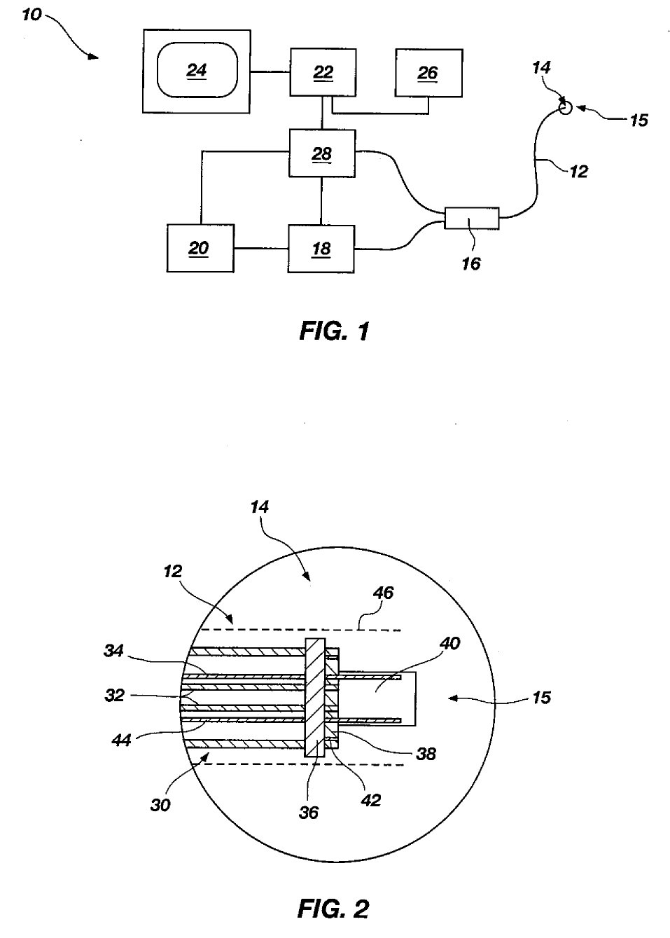

With reference to FIGS. 1 and 2, in one embodiment of the invention, a medical imaging system 10 comprises a micro-guidewire 12 having an imaging device, shown generally at 14, disposed at a distal tip 15 of the micro-guidewire 12. A processor 22, such as an appropriately programmed computer, is provided to control the imaging system 10 and create an image of anatomy adjacent the distal tip portion 15, within a patient (not shown), displayable on a monitor 24, and storable in a data storage device 26. An interface 28 is provided which supplies power to the imaging device 14 and feeds a digital image signal to the processor based on a signal received from the imaging device via an electrical umbilical 30, including conductive wires 32 through the micro-guidewire 12. A light source 44 may also be provided at the distal end of the micro-guidewire.

In one aspect, the system further includes a fitting 16 enabling an imaging fluid, such as a clear saline solution, to be dispensed to the distal tip portion of the micro-guidewire from a reservoir 18 through an elongated tubular member (not shown) removably attached to the micro-guidewire to displace body fluids as needed to provide a clearer image. A pump 20 is provided, and is manually actuated by a medical practitioner performing a medical imaging procedure, or can be automated and electronically controlled so as to dispense fluid on demand according to control signals from the practitioner, sensors, or according to software commands.

With more specific reference to FIG. 2, the imaging device 14 at the distal tip 15 can include a utility guide 36 for supporting or carrying the umbilical 30, which can include electrical wires 32, a fluid dispenser 34, and a light source 44. Other components that can be carried by the utility guide can include, temperature sensors, force sensors, fluid irrigation or aspiration members, pressure sensors, fiber optics, microforceps, material retrieval tools, drug delivery devices, radiation emitting devices, laser diodes, electric cauterizers, and electric stimulators. The utility guide can also carry an SSID or solid state imaging device 38 that includes an imaging array (not shown) and conductive pads 42 for coupling the electrical wires to the SSID. The light source shown is a fiber optic carried by the utility guide. However, other light sources can be used, such as those carried by the SSID. For example, the SSID can also include light-emitting diodes (LEDs) configured to illuminate the area immediately adjacent the distal tip portion. With the SSID in this configuration, a GRIN lens 40 is shown optically coupled to the imaging array of the SSID. In one embodiment of the present invention, the SSID has a maximum width of approximately 450 microns.

The GRIN lens 40 can be substantially cylindrical in shape. In one embodiment, the GRIN lens can have a first flat end for receiving light, a second flat end for passing the light to the imaging array, and an outer curved surface surrounded by an opaque coating or sleeve member to prevent unwanted light from entering the GRIN lens. The GRIN lens can be optically coupled to the imaging array by direct contact between the second flat end and the imaging array of the SSID 38. Such direct contact can include an optically transparent or translucent bonding material at the interface between the second flat end and the imaging array. Alternatively, the GRIN lens can be optically coupled to the imaging array of the SSID through an intermediate optical device, such as a fiber optic or a color filter, or any shape optical lens such as a prism or wide angle lens.

The micro-guidewire 12 can be configured to be bendable and flexible so as to be steerable within a patient's anatomy and to minimize trauma. For example, the micro-guidewire can comprise a micromachined tube 46 at the distal tip portion, and cut-out portions (not shown) can allow for increased flexibility of the tube. Such a micromachined tube can also allow bending to facilitate guiding the micro-guidewire to a desired location by selection of desired pathways as the micro-guidewire is advanced. In one aspect of the invention, the micro-guidewire has a maximum diameter of approximately 760 microns. Additional details on construction of similar slotted micro-machined tube or segments can be found in U.S. Pat. No. 6,428,489, which is incorporated herein by reference.

The micro-guidewire 12 can alternatively comprise an internal tensionable wire (not shown) adjacent one side of the distal tip portion, which when tensioned, causes the distal tip portion 15 to deflect as is known in the art. A combination of deflection and rotation of the distal tip portion of the micro-guidewire provides steer-ability of the device. Another alternative for directability of the distal tip portion is to provide a micro-actuator (not shown) such as an element which expands or contracts upon application of an electrical current signal. Such an element can be substituted for the tension wire, for example.

In another embodiment, the micro-guidewire 12 further comprises a selectively extendable steerable member 34 which may be extended past the distal end of the micro-guidewire and guided into, for example, a secondary body cavity. The selectively extendable steerable member 34 may be viewed by imaging device 14 while simultaneously being steered into a secondary body cavity. Once the selectively extendable steerable member 34 is properly advanced into the secondary body cavity, the micro-guidewire 12 can be advanced into the secondary body cavity. Advantageously, the smaller selectively extendable steerable member may be more easily guided through more tortuous environments thereby facilitating advancement of the entire micro-guidewire 12 assembly through the body.

As will also be appreciated, while the system is illustrated by the exemplary embodiment of a medical imaging system, these arrangements could be used in other devices, such as visual sensors in other devices, surveillance apparatus, and in other applications where a very small imaging device can be useful.

Moreover, with reference to all of the embodiments described herein, the device contemplated can be very small in size, and accordingly the imaging array of the SSID can have a lower pixel count than would otherwise be desirable. As technology advances, pixel size can be reduced, thereby providing clearer images and data. However, when using a lower number of pixels in an imaging array, the resolution of the image provided by the device can be enhanced through software in processing image data received from the SSID. The processor showing in FIG. 1, can be appropriately programmed to further resolve a scanned image from an array of an SSID, for example, based on information received as the SSID is moved slightly, such as from controlled vibration. The processor can analyze how such image data from the imaging array is altered due to the vibration, and can refine the image based on this information.

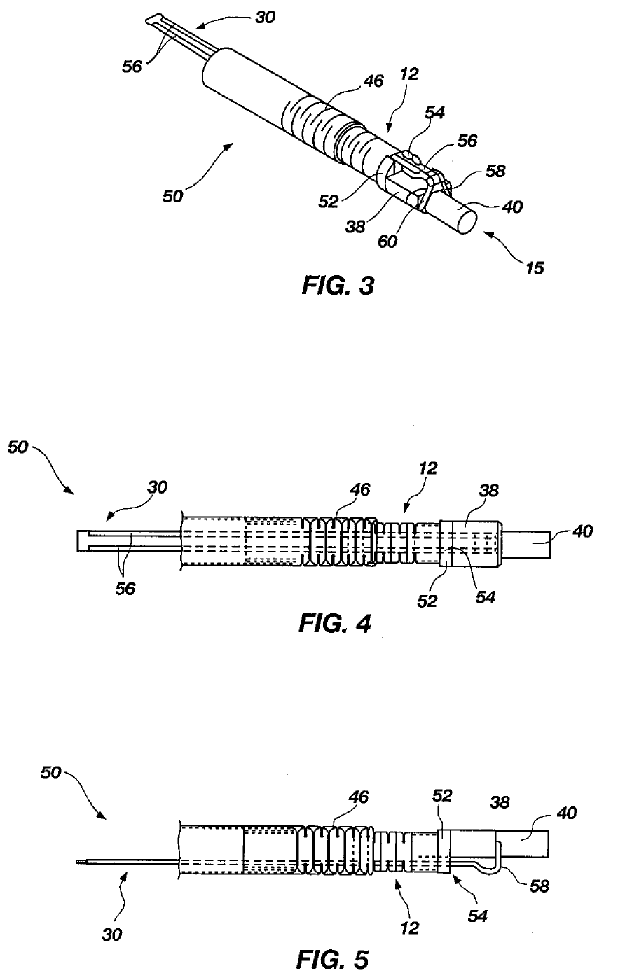

Turning now to FIGS. 3 to 5, another embodiment of the invention is implemented as shown in system 50, wherein a distal tip portion 15 of a micro-guidewire 12 includes lens 40 optically coupled to an SSID 38. Here, the SSID is also electrically bonded to an adaptor 52. The adaptor is carried by micromachined tubing segment 46, and is configured to fit within it at a distal end of the tubing segment. The adaptor has a channel 54 formed therein which allows passage of a conductive strip 56 (which functions similarly as the conductive wires of FIG. 2) of an umbilical 30. The micromachined tubing segment itself is configured to provide telescoping action. This allows the distal tip portion of the micro-guidewire to be assembled and then connected easily to the remainder of the micro-guidewire. The conductive strip can comprise a ribbon formed of a non-conductive material, such as KAPTON, with conductive traces overlain with a dielectric, and provides an electrical umbilical to the SSID through the adaptor. The conductive strip can be threaded back through the micro-guidewire to a fitting (not shown) at its proximal end, as discussed previously. At a distal portion of the conductor strip, individual conductor elements 58, 60 are separated from the non-conductive strip and are bonded to conductive pads (not shown in FIG. 3-5) that are present on the adaptor. Thus, the adaptor provides a power conduit from the umbilical to the SSID. A more detailed description of the use of such an adaptor is described in a U.S. patent application Ser. No. 10/391,513 which is incorporated herein by reference. Alternatively, configurations wherein the SSID and the utility guide are integrated as a single unit are described in an additional U.S. patent application Ser. No. 10/391,490 which is also incorporated herein by reference.

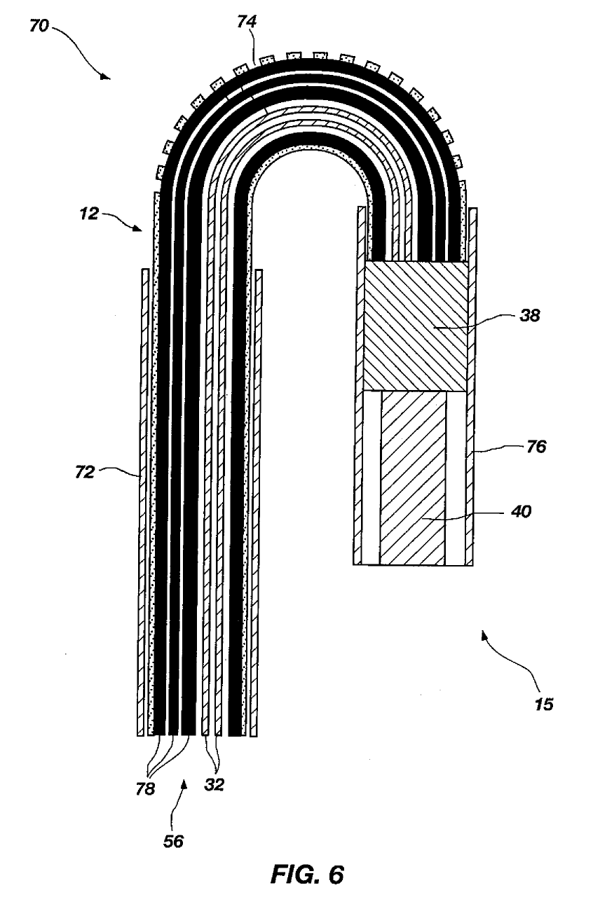

With reference to FIG. 6, another system is shown generally at 70. In this embodiment, the distal tip 15 of the micro-guidewire 12 is shown. An outer sleeve 72 is provided over the outside of the micro-guidewire in telescoping fashion. The micro-guidewire can be withdrawn into the sleeve at will by differential movement at a proximal end (not shown) of the device. An outer tubing of the micro-guidewire can be micromachined to provide a pre-disposition to bend adjacent the SSID 38, for example by micomachining the tubing to provide openings 74 on one side of the tubing and bending the tubing to give it a curved configuration doubling back on itself as shown in the figure. The tip can be directed as desired by pulling the curved portion of the micro-guidewire partially, or completely, back into the outer sleeve. In one embodiment, the micro-machined tubing is formed of super-elastic material with embedded shape memory capability, such as NiTi alloy so that this can be done repeatedly without the material taking a set. A further outer sleeve 76 is provided adjacent the SSID and GRIN lens 40 to support this structure. A conductive strip 56, including conductive wires 32, can be provided, as described previously.

In another embodiment, tensioning wires 78 can be provided in a lumen within the micro-guidewire adjacent a large radius, or outer portion of the micro-guidewire 12, which enables directing the tip 15 by providing a tension force tending to straighten out this portion of the micro-guidewire. The tension wire is attached to the SSID 38 and extends back through the micro-guidewire to a proximal portion where it can be manipulated by a practitioner doing the imaging procedure. The micro-guidewire can also include provision for supplying imaging fluid, light, or other utilities, as discussed above.

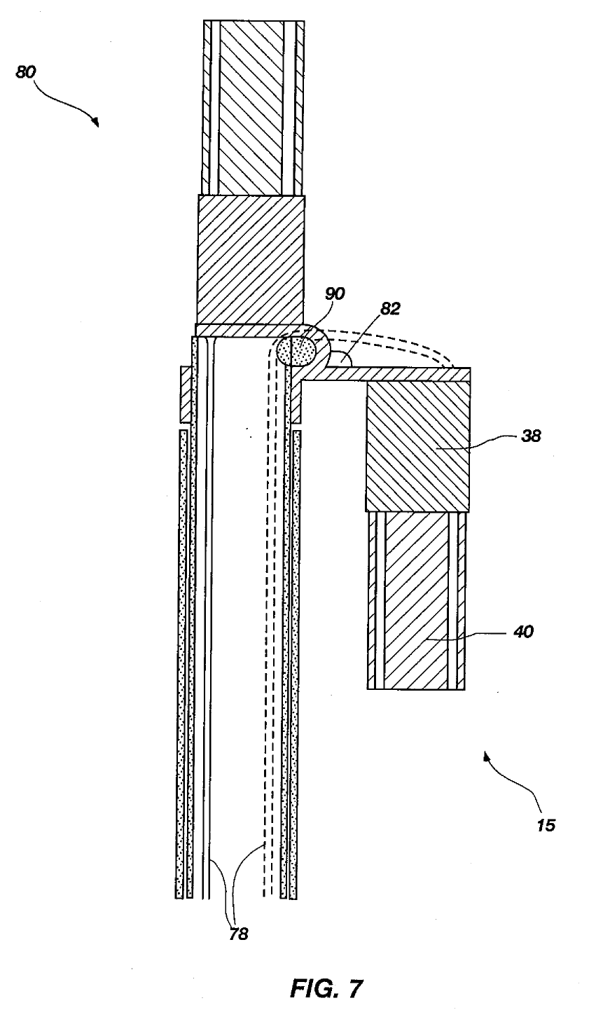

With reference to FIG. 7, a system shown generally at 80, can comprise an SSID 38 mounted on a hinge 82 formed of super-elastic material with embedded shape memory capability. Tensioning wires 78 are attached to the hinge, and allows the SSID to be directed from a first direction aimed back along the longitudinal axis of the micro-guidewire, through 180 degrees, to a second position aiming distally away from the micro-guidewire in a direction substantially coincident with the longitudinal axis. This, in combination with rotation of the micro-guidewire, allows for directability of the tip. A rounded guide 90 is attached to a distal portion of the tube to provide a radius for the tensioning wires and the hinge so that they do not kink, but deform elastically as shown. Conductive wires (not shown) can be present as describe previously.

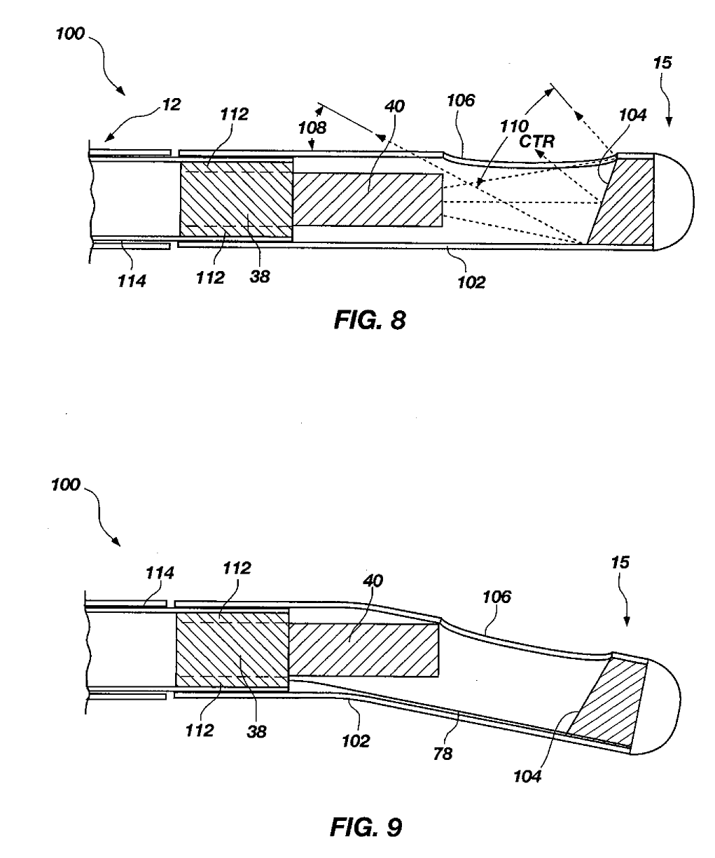

Continuing now with reference to FIGS. 8 and 9, an alternative system is shown generally at 100. As shown, control means for directing the micro-guidewire 12 and/or directing the field of view of the SSID 38 at the distal tip portion 15 of the micro-guidewire is illustrated. A deformable outer sleeve 102 comprising a mirror element 104 at a distal end is provided. An opening 106 adjacent the mirror element and the GRIN lens 40 enables appropriate imaging.

In one configuration state, shown in FIG. 8, the angled surface of the mirror allows a view rearwardly and to the side of the micro-guidewire at an angle 108 of about 25 to 50 degrees with respect to a longitudinal axis of the micro-guidewire. A field of view 110 based on the configuration and spacing, and angular relationships between the elements can comprise between about 15 and 25 degrees. The SSID can comprise one or more lumens 112 for conveying imaging fluid to the distal tip portion of the micro-guidewire, or to carry power to the imaging array (not shown) of the SSID. As will be appreciated, imaging fluid could also be conveyed to the imaging site via another lumen 114 or a guiding micro-guidewire, or a completely separate micro-guidewire (not shown).

In another configuration state, shown in FIG. 9, the deformable outer sleeve 102 is bent, enabling direct viewing forwardly through the opening 106. Also, views rearwardly at various angles can be obtained by causing more or less deflection of the deformable outer sleeve 102. Attached to the tube adjacent one side (a bottom side in FIG. 9), a tension wire 78 deflects the deformable outer sleeve as tension is applied. Another way for deforming the sleeve is to form it from a NiTi alloy, which changes shape from a first configuration shown in FIG. 8 to a second configuration in FIG. 9 via change of temperature such as can be affected by introduction of imaging fluid of a different temperature, or by running an electrical current therethrough. In the latter two embodiments, the tip has essentially two states, deformed and undeformed.

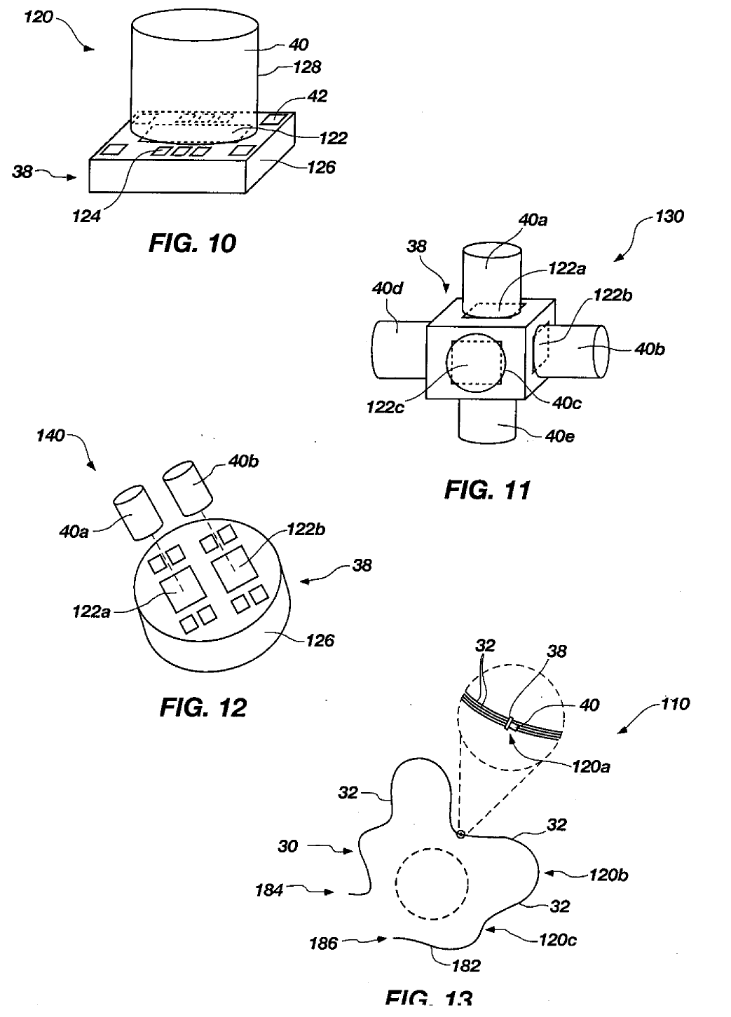

Referring now to FIG. 10, a system, indicated generally at 120, includes a GRIN lens 40 and an SSID 38. The SSID can comprise a silicon or silicon-like substrate or amorphous silicon thin film transistors (TFT) 126 having features typically manufactured therein. Features including the imaging array 122, the conductive pads 42, metal traces (not shown), circuitry (not shown), etc., can be fabricated therein. With respect to the conductive pads, the connection between conductive pads and a conductive line of an umbilical (not shown) can be through soldering, wire bonding, solder bumping, eutectic bonding, electroplating, and conductive epoxy. However, a direct solder joint having no wire bonding between the electrical umbilical and the conductive pads can be preferred as providing good steer-ability and can be achieved with less risk of breaking electrical bonding. In one embodiment, the conductive line of the umbilical can provide power, ground, clock signal, and output signal with respect to the SSID. Other integrated circuit components can also be present for desired applications, such as light emitting diodes (LEDs) 124, for providing light to areas around the GRIN lens.

It is not required that all of these components be present, as long as there is a visual data gathering and sending image device present, and some means provided to connect the data gathering and sending device to a visual data signal processor. Other components, such as the umbilical, housing, adaptors, utility guides, and the like, can also be present, though they are not shown in FIG. 10. The SSID 38 can be any solid state imaging device, such as a CCD, a CID, or a CMOS imaging device. Also shown, the GRIN lens 40 is coated with an opaque coating 128 on the curved surface to prevent light from entering the lens at other than the flat surface that is most distal with respect to the SSID.

FIG. 11 depicts an alternative system 130 that includes multiple imaging arrays 122a, 122b, 122c on a common SSID 38. Though only three imaging arrays are shown in this perspective view, five imaging arrays are present in this embodiment (i.e., one on each side of five sides the substrate 126, with the back side of the substrate providing a surface for umbilical connection). Each imaging array is respectively optically coupled to a GRIN lens 40a, 40b, 40c, 40d, 40e. As can be appreciated, this is but one configuration where multiple imaging arrays with multiple GRIN lenses can be used. Fewer or more imaging arrays can be used in other similar embodiments, and/or can be part of multiple SSIDs. Umbilical connections are not shown, though it is understood that an umbilical can be present to operate the SSID and its multiple imaging arrays (either by signal splitting or by the use of separate power and/or signal sources).

FIG. 12 depicts a system, shown generally at 140, which can provide stereoscopic imaging. Specifically, multiple imaging arrays 122a, 122b, are shown on a common SSID 38 in a coplanar arrangement. A pair of GRIN lenses 40a, 40b are shown as they would be optically coupled to imaging arrays 122a, 122b, respectively. Other than the imaging array, other features are also present in the SSID, including conductive pads for providing an electrical connection to an umbilical (not shown).

Referring now to FIG. 13, a system 110 includes multiple microcameras 120a, 120b, 120c positioned along an umbilical 30, which are attached to conductive wires 32 of the umbilical. The umbilical includes a proximal end 184, which can be coupled to a processor/monitor (not shown) for viewing, and a distal end 186. Each microcamera includes an SSID 38 and a GRIN lens 40. In the embodiment shown, the microcamera 120c that is closest to a terminal end 186 is optically coupled to a fiber optic line 182, which can include a GRIN lens at a terminal end of the fiber optic line, as shown in FIG. 18 below. However, the microcamera closest to the terminal end can actually be at a distal tip of the micro-guidewire.

The embodiments thus far shown depict GRIN lenses optically coupled to imaging arrays of SSIDs by a direct bonding or coupling. However, the term “optically coupled,” also provides additional means of collecting light from GRIN lens and coupling it to an imaging array of an SSID. For example, other optical devices can be interposed between a GRIN lens and an SSID, such as a color filter, fiber optic, or any shape optical lens including a prism or wide angle lens. Specifically, a system of converting monochrome imaging to multiple colors can be accomplished by utilizing a filter having a predetermined pattern, such as a Bayer filter pattern. The basic building block of a Bayer filter pattern is a 2×2 pattern having 1 blue (B), 1 red (R), and 2 green (G) squares. An advantage of using a Bayer filter pattern is that only one sensor is required and all color information can be recorded simultaneously, providing for a smaller and cheaper design. In one embodiment, demosaicing algorithms can be used to convert the mosaic of separate colors into an equally sized mosaic of true colors. Each color pixel can be used more than once, and the true color of a single pixel can be determined by averaging the values from the closest surrounding pixels.

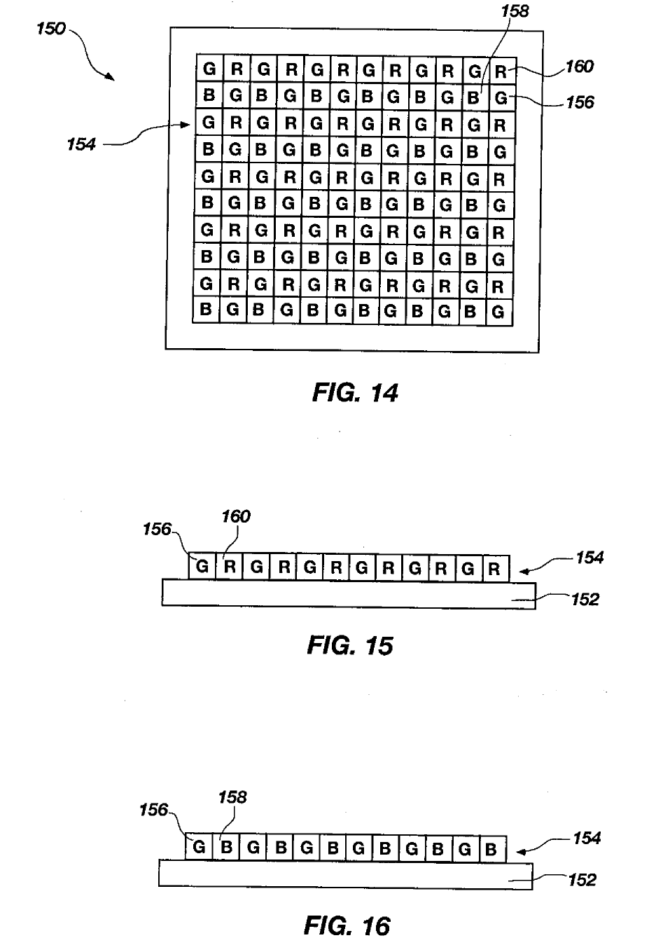

Specifically, with reference to FIGS. 14-16, a color filter insert, shown generally at 150, can comprise a substantially optically clear filter substrate 152 and a color filter mosaic portion 154. The filter insert as a whole is made up of green transparent color material 156, blue transparent color material 158, and red transparent color material 160. Each of the transparent color material 156, 158, 160 can be polymerized color resins such as those available from Brewer Science. In one embodiment, the green color material 156 can be put down on the clear filter substrate first, and then the red 160 and blue 158 color material can be positioned in the appropriate spaces provided by the green material. Each transparent color material can be configured to be the size of an SSID image array pixel. The optically clear filter substrate can be, for example, a polymeric material such as SU-8 available from IBM, having a thickness of about 20 microns, though other thicknesses and materials can be used.

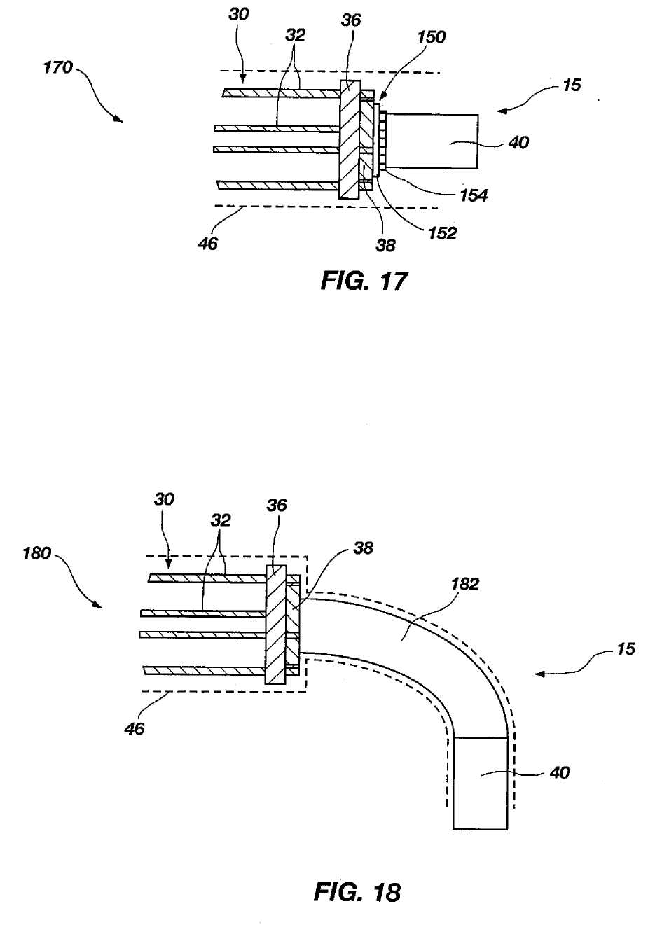

Turning now to FIG. 17, a system 170, including a color filter insert 150 having an optical clear filter substrate 152 and the color filter mosaic portion 154, can be positioned between a GRIN lens 40 and an imaging array (not shown) of an SSID 38. FIG. 18 depicts an alternative system 180, wherein a fiber optic 182 is used to optically couple a GRIN lens 40 with an imaging array (not shown) of an SSID 38. Any bonding technique or mechanical coupling can be used to connect the SSID to the GRIN lens through the color filter insert or fiber optic in order to make the optical connection, such as bonding by an optically clear bonding epoxy. In both FIGS. 17 and 18, as described previously, the imaging device at the distal tip 15 can include a utility guide 36 for supporting or carrying the umbilical 30, which can include electrical wires 32 and other utilities (not shown). Both FIGS. 17 and 18 also depict micromachined tubing 46 to support and direct the camera.

As will be appreciated, an imaging device in accordance with principles of the invention can be made very small, and is useful in solving certain imaging problems, particularly, that of imaging a remote location within or beyond a small opening, for example in human anatomy distal of a small orifice or luminal space (anatomical or artificial, such as a trocar lumen), or via a small incision, etc. In fact, because of the solid state nature of the SSID, and because of the use of the GRIN lens, these cameras can be made to be micron-sized for reaching areas previously inaccessible, such as dental/orthodontics, fallopian tubes, the pancreatic duct, heart, lungs, vestibular region of ear, and the like. Larger lumens or cavities can be viewed with a greater degree of comfort and less patient duress, including the colon, stomach, esophagus, or any other similar anatomical structures. Additionally, such devices can be used for in situ tissue analysis.

Previous attempts to obtain internal body images have been focused on the use of endoscopes and large micro-guidewire devices whose access potential is principally limited to the primary paths of the body. Such primary paths can include: the esophagus, stomach, and colon. What has been done with the present invention is to advance the imaging potential of such devices into the secondary paths of the body, such as the fallopian tubes, pancreatic duct, common bile duct, bronchioles of the lungs, and so forth. Among the reasons for this advancement are 1) the size of the present invention and 2) the steer-ability that is provided by the small size and flexibility of the umbilical body, as previously described. These features have provided the capability of being able to direct the microscopic camera and umbilical body into small openings, orifices, lumens, incisions, etc.

One embodiment of the miniature imaging device of the present invention, that includes a CCD camera and a GRIN lens, can be literally diverted from a primary path of the body to a secondary path and continue the penetration into the secondary, much smaller, path by directing the camera and advancing the camera from the proximal end of the micro-guidewire. By thus being able to maneuver and identify environmental elements within a body we can image critical organs of the body that have hereto been inaccessible with a single micro-guidewire inserted into a body cavity.

FIG. 28 depicts a miniature imaging device 200, such as a micro-guidewire, according to the present invention being directed along a primary path 320 within a living body. The micro-guidewire can include an imaging device disposed on the distal end of the micro-guidewire. As described in detail above, the imaging device can include a SSID including an imaging array and a GRIN lens optically coupled to the imaging array of the SSID. Branching from the primary path is a secondary path 322 of the body. The secondary path is much smaller. For example, the secondary path can have a diameter of approximately ½ to approximately 1/20 times the size of the primary path diameter. The micro-guidewire 200 can identify the secondary path that laterally branches from the primary path. The miniature imaging device can then be turned into the secondary path and be advanced by applied pressure at the proximal end of the micro-guidewire.

Primary paths of the body can include the esophagus, colon, ear canal, intestines, trachea, urethra, and other bodily paths that are accessible via a bodily orifice, as well as paths that are easily accessible from these paths. For example, a micro-guidewire can be inserted into a primary path such as the esophagus via the mouth. By directing and advancing the micro-guidewire further into the body a user can direct the micro-guidewire through the stomach cavity into the duodenum of the small intestine. This continual path constitutes a primary path of the body. In the case that a user intends to image the pancreas, the user must identify the ampulla of Vater and turn the distal end of the micro-guidewire from the primary path into this secondary path of the duodenum. Similarly, by directing a micro-guidewire down the primary path of the trachea a user can reach the primary branches of the trachea into the lungs. By advancing the micro-guidewire further into the lungs a user will be required to identify target paths or secondary paths of the lungs and turn the distal end of the micro-guidewire into these paths.

Previous attempts to direct micro-guidewires into secondary paths of the body have been limited by the steer-ability, size and flexibility of the micro-guidewire or endoscope used. In one embodiment of the present invention, the micro-guidewire is advanced down a secondary path wherein the secondary path has a maximum diameter of about 800 micrometers. As described above, the steer-ability, flexibility, and miniature-size provided by the present invention allow the miniature imaging device to be literally diverted from a path, going in the direction of the longitudinal axis of the device, into a secondary path positioned at an angle greater than 60-degrees off the axis of the primary path. Previous attempts to turn a miniature device at such a sharp angle into such a small diameter opening have failed due to the lack of flexibility and steerability that these devices afforded a user.

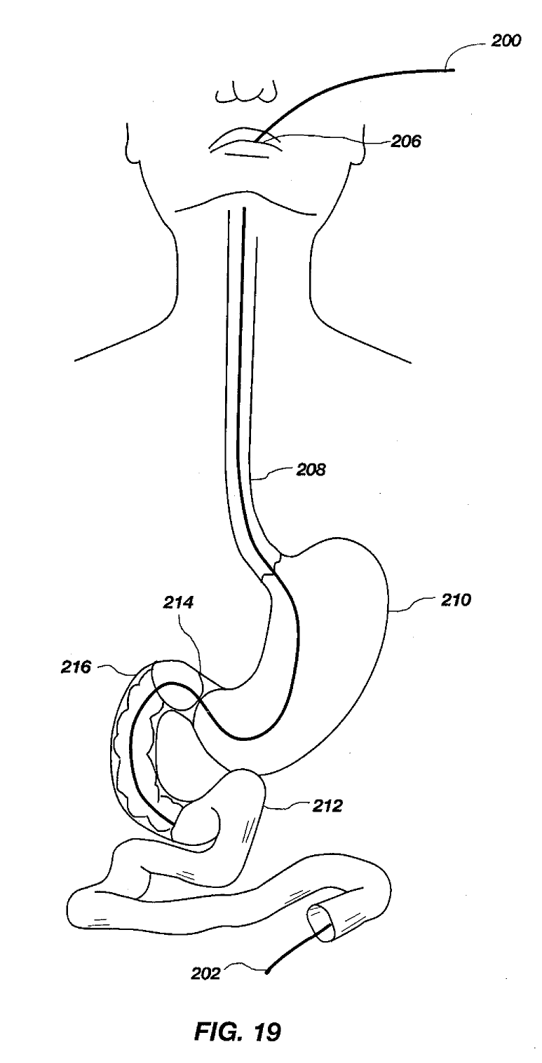

FIG. 19 depicts an exemplary miniature imaging device 200 that can be used to image the gastrointestinal tract, which can include performing an esophagogastroduodenoscopy (EDG/OGD) and/or eneteroscopy. The micro-guidewire or miniature imaging device 200 is inserted into the mouth 206 and directed down the esophagus 208 through the stomach 210 and pylorus 214 to examine the duodenum 216, jejunum 212 and ileum (not shown) of the small intestine. The micro-guidewire, can be advanced by applying pressure to the proximal end of the micro-guidewire. As described above, the micro-guidewire body can include an umbilical. By turning and/or bending the distal end of the micro-guidewire the micro-guidewire can then be directed along the primary paths of the body with minimal trauma. The micro-guidewire can include a utility guide for carrying utilities, as described above. This utility guide can include an injection needle for injecting a liquid at target locations, electric cauterizers, micro forceps, as well as a snare and/or other devices that will be apparent to one of skill in the art.

Esophagogastroduodenoscopy, or an endoscopy of the upper gastrointestinal tract, has typically included the risk of causing bleeding and/or perforation of the organs and other tissue by an endoscope. Due to its size and flexibility, the micro-guidewire of the present invention can reduce these risks and provide a greater degree of comfort to the patient. Additionally, a deeper and more advanced diagnosis is possible due to the variable length of the umbilical and the high resolution CCD cameras having a GRIN lens.

Eneteroscopy, or an endoscopy of the small intestine, has posed a challenge to gastroenterologists due to the difficulty of physically reaching and imaging the small bowel anatomically. Long gastroscopes or colonoscopes have been employed for visualizing the jejunum 212, or middle portion of the small intestine. These scopes are typically large in diameter and can cause a patient discomfort and duress. In addition to the discomfort historically involved with endoscope imaging, this process has typically provided only marginal image resolution due to the image quality that is lost by using a fiber optic cable for transmitting image data from the imaging site to the remote processor and display. To increase patient comfort and reduce the risk of internal trauma, wireless capsule endoscopy can be used to visualize the gastrointestinal tract. However, wireless capsule endoscopes are limited by intermittency of images and inability to obtain biopsies. The micro-guidewire 200 of the present invention can overcome the drawbacks of these prior art approaches while retaining a high degree of image resolution while decreasing the risk of patient trauma and discomfort.

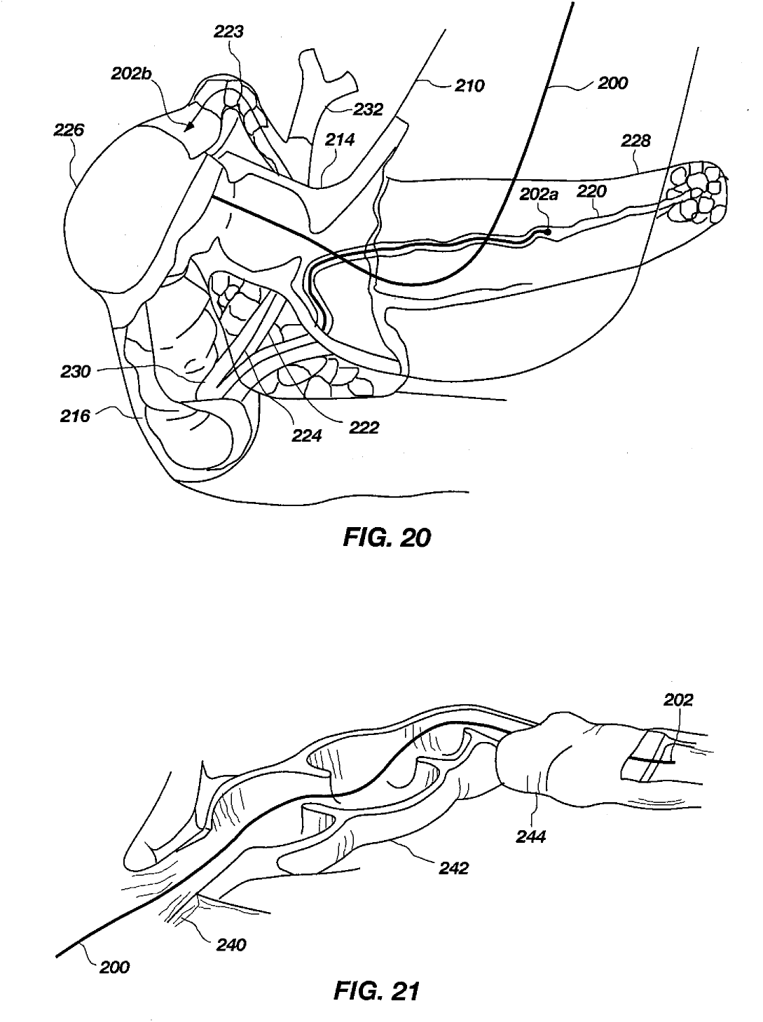

Turning now to FIG. 20, the micro-guidewire 200 can be used to image the biliary tree of the liver, the gallbladder 226, and the pancreatic duct 220. Up to now the only way to image these secondary tracts involved the use of x-ray imaging combined with an endoscope having an auxiliary extendible micro-guidewire, in a procedure known as endoscopic retrograde cholangiopanreatography (ERCP). This process can now be accomplished with the micro-guidewire of the present invention in a much simpler and less traumatic procedure.

As explained above, the micro-guidewire 200 can be inserted through the mouth and directed to the duodenum 216 of the small intestine. Here the micro-guidewire can identify the ampulla of Vater, the opening in the duodenum into the common bile duct 222 and the pancreatic duct 220. The ampulla of Vater is a small opening that branches laterally from the duodenum. The distal end of the micro-guidewire can be turned into the ampulla of Vater whereupon this ampulla branches into the pancreatic duct and the common bile duct. As described above, the micro-guidewire can be advanced into either of these ducts by an applied pressure at the proximal end of the micro-guidewire.

Through the common bile duct a physician can enter the cystic duct 223 to image the gallbladder or the common hepatic duct 232 to image the liver. The micro-guidewire 200 can include various devices associated with or on the utility guide for treating and diagnosing the gallbladder and liver. For exemplary purpose, the treatment of the gallbladder will be described herein. The utility guide can include a balloon that when inflated can expand the bile duct and allow the passage of gallstones. Laser diodes can be included on the utility guide to break gallstones into pieces in order to facilitate removal. Electrical stimulators can also be used to enlarge the ampulla and other sphincters. Other devices can be included to assist in the drainage of bile and other necessary procedures.

Pancreatitis, pancreatic cancer, and other pancreas-related diagnosis can be enhanced and facilitated when high resolution imaging is utilized, as with the micro-guidewire 200 of the present invention. Pantreatic cancer is represented by a growth of a malignant tumor within the pancreas. Historically, once the symptoms are able to be recognized and diagnosed, the cancer is advanced and difficult to treat. The miniature imaging device of the present invention can facilitate the early detection of this disease by allowing a practitioner to image directly into the pancreas in order to detect the early stages cancer.

FIG. 21 depicts a micro-guidewire 200 that has been inserted into the anus 240 and advanced to image the rectum 242 and colon. Typical colonoscopies utilize an endoscope that can cause discomfort and internal trauma. By utilizing the micro-guidewire 200 of the present invention the procedure can be much less invasive, such that a patient would not even have to be asleep. A complete examination of the colon, which can measure over six feet in length, can be accomplished with this single steerable micro-guidewire. Additionally, the vermiform appendix (or simply, the appendix) which stems from the large intestine could be imaged by the micro-guidewire that is inserted in the anus and directed through the large intestine. This less-invasive procedure could provide medical practitioners a procedure for imaging the appendix without the need of small incisions which are typically required in a laparoscopic procedure for viewing the appendix.

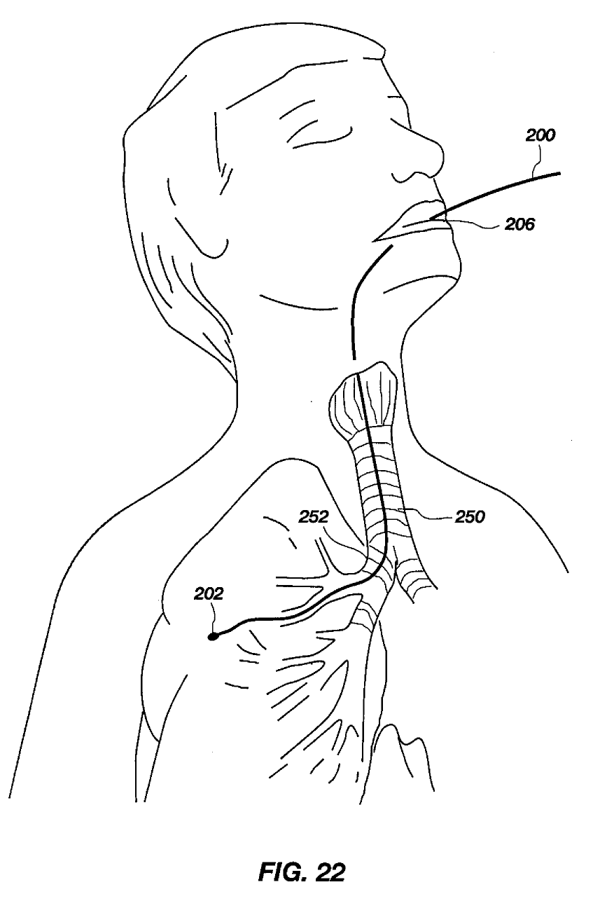

Referring now to FIG. 22, a micro-guidewire 200, according to the present invention, can be capable of imaging small branches of the lungs. The micro-guidewire can be inserted into the mouth 206 of a patient and directed down the trachea 250 to the right 252 or left bronchus. From this primary path the micro-guidewire can be directed and advanced into the bronchial tubes, for example the tertiary bronchus tubes and bronchioles. This analysis can allow a doctor to recognize early stages of various lung diseases, such as small cell carcinoma, adenocarcinoma, various forms of lung cancer, and other like problems and diseases.

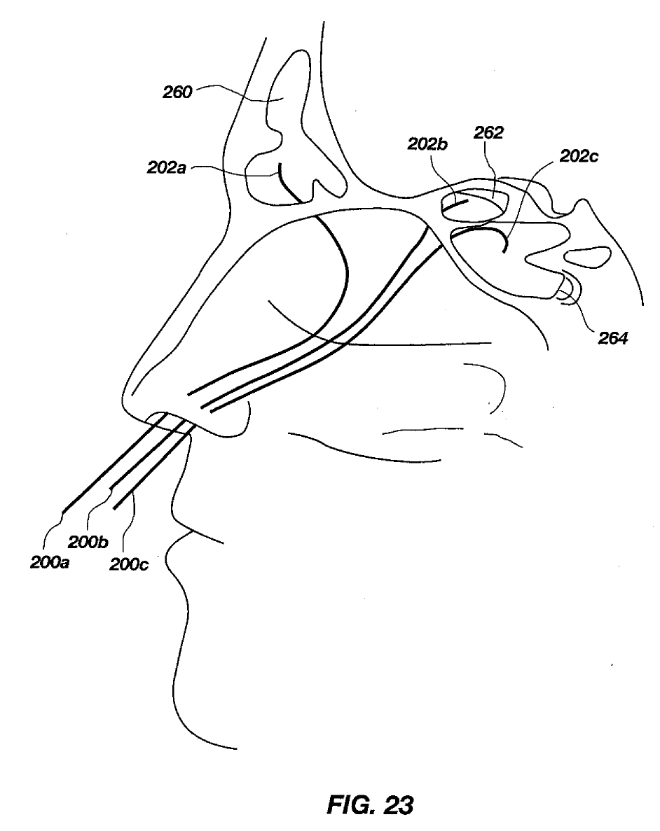

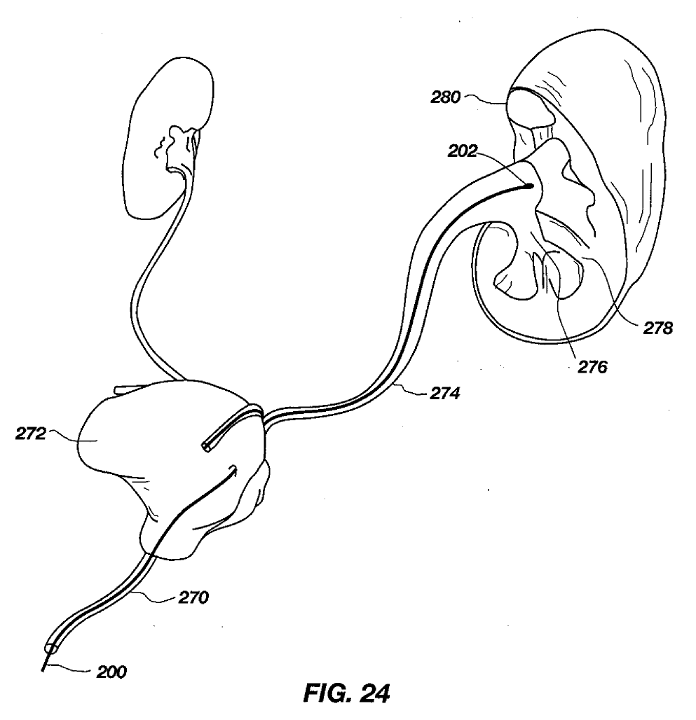

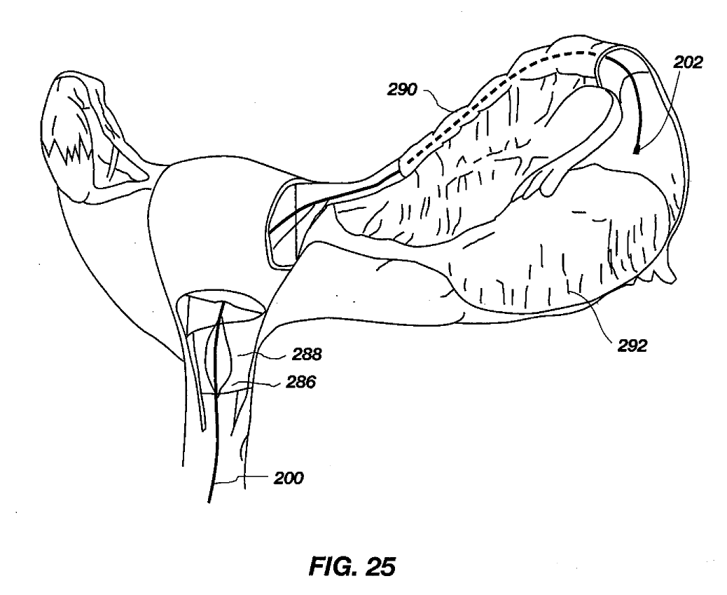

FIG. 23 depicts three paths of a micro-guidewire represented as 200a, 200b, and 200c, with heads 202a, 202b, and 202c respectively. By inserting a micro-guidewire into the nasal cavity and directing the micro-guidewire through one of the patient's paranasal sinuses 260, 262, 264, a doctor can diagnose structural defects, infection or damage to the sinuses, or structures in the nose and throat. A drug delivery system can be included on a utility guide for supplying a drug to treat sinus infections, sinusitis, and other such ailments.

Turning now to FIG. 24, a micro-guidewire 200 according to one embodiment of the present invention can also be capable of inspecting the ureters and kidneys. The micro-guidewire can be inserted into the urethra 270 and directed into the bladder 272. Once in the bladder the micro-guidewire can be directed to a ureter 274 and advanced up into a kidney 280. This small micro-guidewire can image tissue within the major and minor calyx of the kidneys, as well as assist in removing or breaking apart kidney stones.

FIG. 25 depicts a micro-guidewire 200 that is capable of inspecting the fallopian tubes and the ovaries. The micro-guidewire can be inserted through the cervical canal 286 and uterine cavity 288 into the fallopian tubes 290. The micro-guidewire can inspect the fallopian tubes as well as be directed through the fallopian tubes to image the ovaries 292. A difficult problem in the field of medical diagnosis of ovarian cancer is that often a visual inspection of the ovary is required to confirm a diagnosis of ovarian cancer. This conventionally implicates a surgical or laparoscopic procedure under general anesthesia, with attendant cost and risk. It also implies that the medical practitioner should be prepared to treat the cancer, surgically or otherwise, in the same procedure upon diagnosis of ovarian cancer, so that a subsequent procedure can be avoided. However, these downfalls and their attendant costs can be avoided by directly imaging the ovaries with the miniature endoscope of the present invention. Additionally, the utility guide can include microforceps and other miniature surgical devices to perform various procedures using the miniature imaging device.

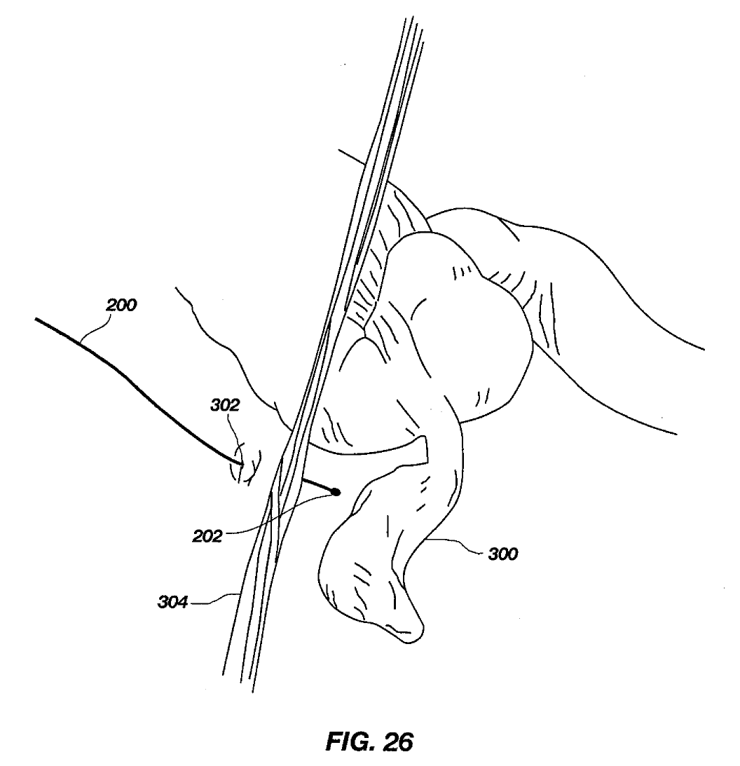

FIG. 26 depicts a micro-guidewire 200, according to the present invention, performing a laparoscopic procedure. While the present figure and description is directed towards visualization of the appendix 300, it will be noted that the invention is not limited solely to this single laparoscopic procedure but can be used for all such laparoscopic procedures known in the art. Because of the decreased size of the micro-guidewire 200 of the present invention the numerous advantages of using a GRIN lens with a CCD camera, such a laparoscopic procedure can be facilitated and enhanced. A smaller incision 302 can be made in the skin 304 of a patient for inserting the micro-guidewire. Likewise, the CCD camera with GRIN lens can produce enhanced camera images by using a smaller camera. Once inserted, the micro-guidewire can be directed towards a target location and advanced by pressure at the proximal end of the micro-guidewire. Because of its enhanced steer-ability and flexibility the micro-guidewire can be directed to view remote areas of the body and hereto inaccessible locations.

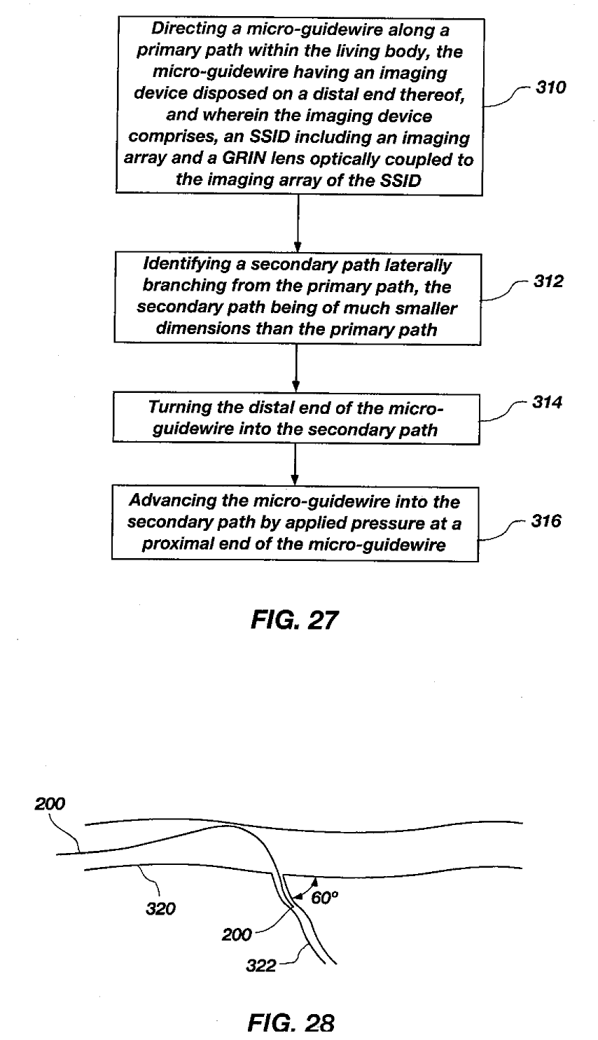

FIG. 27 shows a flowchart for a method for imaging within a living body. First, the method includes directing a micro-guidewire along a primary path of the living body, the micro-guidewire having an imaging device disposed on a distal end of the micro-guidewire, and wherein the imaging device comprises, an SSID including an imaging array and a GRIN lens optically coupled to the imaging array of the SSID 310. This primary path can include the esophagus, small intestines, colon, etc. Next, the method can include identifying a secondary path laterally branching from the primary path, the secondary path being of much smaller dimensions than the primary path 312. This secondary path can be one of the exemplary paths given in the above described figures, or any other secondary path of the body that branches from a primary path. The method can then include turning the distal end of the micro-guidewire into the secondary path 314. This turning can be performed by a number of distinct methods and apparatuses, including the apparatuses shown in FIGS. 6-9. The method further includes advancing the micro-guidewire into the secondary path by applied pressure at a proximal end of the micro-guidewire 316. Because the micro-guidewire of the present invention is highly flexible and steer-able it can be driven solely by pressure applied to its proximal end.

It is to be understood that the above-referenced arrangements are illustrative of the application for the principles of the present invention. Numerous modifications and alternative arrangements and procedures can be devised without departing from the spirit and scope of the present invention while the present invention has been shown in the drawings and described above in connection with the exemplary embodiments of the invention. It will be apparent to those of ordinary skill in the art that numerous modifications and alternative endoscopic procedures can be made and performed without departing from the principles and concepts of the invention as set forth in the claims.

1. An apparatus for imaging a portion of a body cavity, comprising:
a steerable micro-guidewire having a maximum diameter of approximately 760 microns;
an SSID including an imaging array disposed on a distal end of the micro-guidewire; and
a lens system disposed on a distal end of the SSID.

2. The apparatus of claim 1, wherein the lens system comprises a GRIN lens bonded directly to the imaging array of the SSID, the GRIN lens having a first flat surface and a second flat surface.

3. The apparatus of claim 2, wherein the GRIN lens is bonded to the imaging array of the SSID at the first flat surface of the SSID and the first flat surface of the GRIN lens.

4. The apparatus of claim 1, wherein the steerable micro-guidewire further comprises a semi-rigid proximal end and a flexible distal end, the flexible distal end having a plurality of machined cuts disposed on an outer portion of the micro-guidewire.

5. The apparatus of claim 1, wherein the steerable micro-guidewire further comprises an elongated hollow tubular member removably attached thereto.

6. A medical device, comprising:
a flexible terminal segment of a steerable micro-guidewire having a plurality of machined cuts disposed on an outer portion thereof;
an SSID including an imaging array disposed on a distal end of the flexible terminal segment, the SSID having a maximum width of about 450 microns; and
a lens system disposed on a distal end of the SSID.

7. The medical device of claim 6, wherein the micro-guidewire further comprises a utility guide.

8. The medical device of claim 6, further comprising a light source originating from and disposed on the distal end of the flexible terminal segment.

9. The medical device of claim 8, wherein the light source is a light emitting diode.

10. The medical device of claim 6, wherein the micro-guidewire further comprises a steerable member selectively extendable from a distal end of the micro-guidewire.

11. A method of imaging a portion of a body cavity, comprising:
advancing an SSID positioned on at least a portion of a micro-guidewire into a cavity of a body, wherein the SSID includes an image array disposed on a distal end thereof and a lens system disposed on a distal end of the SSID; and
electronically generating image data from the SSID corresponding to at least a portion of the cavity of the body.

12. The method of claim 11, further comprising the step of transmitting the generated image data to a data reception device.

13. The method of claim 11, further comprising the step of processing the image data into a displayable image and displaying an image on a display device.

14. The method of claim 13, wherein a direction of movement within the cavity of the body is a primary path of advancement, the method further comprising the step of identifying a secondary path branching from the primary path as part of a field of view of the SSID, wherein the secondary path has a maximum diameter of about 800 micrometers.

15. The method of claim 14, further comprising the step of advancing the SSID and micro-guidewire into the secondary path branching from the primary path, while viewing the field of view in real-time via the image data transmitted from the SSID.

16. The method of claim 15, wherein the step of advancing includes abruptly diverting the SSID from the primary path to the secondary path oriented on an angle greater than 60 degrees from an axis of the primary path relative to a longitudinal axis of the micro-guidewire.

17. The method of claim 15, wherein the secondary path is oriented on an angle less than 120 degrees from an axis of the primary path relative to a longitudinal axis of the micro-guidewire.

18. The method of claim 13, further comprising the step of advancing a portion of a catheter over a portion of the micro-guidewire.

19. The method of claim 13, further comprising the step of advancing a medical device over a portion of the micro-guidewire while viewing real-time image data on an image device.

20. The method of claim 13, further comprising the step of performing a surgical procedure while concurrently viewing real-time image data on the display device.

21. The method of claim 14, further comprising the step of advancing the micro-guidewire into the secondary path while viewing the secondary path in real-time on the display device.
